(19) United States
(12) Patent Application Publication
(10) Pub. No.: US 2007/0212339 A1
(43) Pub. Date: Sep. 13, 2007

(54) Use Of At Least One Effector Of Glutathione Metabolism Together With Alpha-Lipoic Acid For The Treatment Of Chronically Obstructive Lung Diseases

(75) Inventors: Siegfried Ansorge, Hohenwarthe (DE); Dieter Koegst, Wahlitz (DE); Michael Taeger, Heinrichsberg (DE); Gerhard Fries, Wahlitz (DE)

Correspondence Address: MILLEN, WHITE, ZELANO & BRANIGAN, P.C., 2200 CLARENDON BLVD., SUITE 1400, ARLINGTON, VA, 22201, US

(73) Assignees: ESPARMA GMBH, Osterweddingen (DE); IMTM GMBH, Magdeburg (DE)

(21) Appl. No.: 10/584,072

(22) PCT Filed: Dec. 23, 2004
(86) PCT No.: PCT/EP04/14687

§ 371 Date: Apr. 3, 2007

(30) Foreign Application Priority Data
Dec. 23, 2003 (DE) 103 60 954.7

(51) Int. Cl. A61K 38/16; A61K 31/385

(52) U.S. Cl. 424/94.1; 514/440

(57) ABSTRACT

The present invention relates to the use of at least one effector of glutathione metabolism together with α-lipoic acid, its salts and/or its pro-drugs for the simultaneous, separate or timed cytoprotective treatment of chronically obstructive lung diseases.

Use of at least one effector of glutathione metabolism together with α-lipoic acid for the treatment of chronically obstructive lung diseases

The invention relates to the use of at least one effector of glutathione metabolism together with α-lipoic acid, its salts and/or its pro-drugs for the simultaneous, separate or timed cytoprotective treatment of chronically obstructive lung diseases.

Chronically obstructive lung diseases (chronic bronchitis, chronic obstructive pulmonary diseases COPD) count as the numerically most strongly growing health problems of modern industrial nations. There are various causes for this increase, environmental factors and disadvantageous lifestyles including nicotine abuse playing a particular role. The economic damage arising annually from the direct and indirect costs of illness represents a considerable burden and is the reason for various measures for therapeutic and preventive interventions.

Once chronic lung obstructions have become manifest the causes of them are generally not responsive to therapy. The treatment must be directed to as extensive as possible a reduction of the symptoms. These include secretolysis and bronchodilatation. With the exception of milder forms, an accompanying anti-inflammatory therapy with corticosteroids is mandatory.

In past years, a number of experiments have been carried out in which an attempt was made to trace pathophysiological connections in respect of restriction of lung function and cellular effector mechanisms both of lung cell types and of immigrating or resident immune cells.

As a result, in particular in addition to tissue-destroying processes which are caused by matrix-bound and soluble proteases and ultimately lead to the occurrence of pulmonary emphysema, a dysregulation of the function of typical immune cells of the lung was recorded. To the fore here are alveolar macrophages which, at over 83%, make up the numerically largest proportion of the immune cells in the bronchoalveolar area. Comparing the alveolar macrophages of healthy people with COPD patients shows a clearly reduced functionality of the alveolar macrophages in the patient groups which is characterised mainly by the loss of the original capacity for phagocytosis as well as of bactericidal properties and is regularly accompanied by a defect in the homeostasis of the cytokine production.

Furthermore it was able to be demonstrated that alveolar macrophages of COPD patients and especially of smoking COPD patients have a highly damaged thiol-disulphide status. In conjunction with the knowledge of a direct correlation between thiol deficiency and dysfunction in other cellular systems, the conclusion was obvious that this pulmonary thiol deficiency represents a pathophysiological key role in the occurrence and especially the maintenance of the disease.

Finely regulating the thiol-disulphide status represents one of the most important basic premises of biological metabolism efficiency. The central regulating element within this system is tripeptide glutathione which achieves relatively high intra-cellular concentrations (up to 10 mM) in reduced form.

In addition to glutathione, proteins bearing intra-cellular SH (thiol) groups, and especially such groups in cell membrane-bound form, are further important structural units of the thiol-disulphide status of each cell.

The metabolism of disulphide cleavage and thiol group formation regulated by various enzyme classes must necessarily be intact for any normal cell functioning, due to the variety of its biological functions inter alia in cellular growth and differentiation processes including programmed cell death as well as cell protection and detoxification mechanisms. Disturbances in this system and alterations in the concentration of the thiols lead to serious cellular dysfunctions which only remain locally limited in an isolated case, but generally impair the entire organism.

Thus from DE 101 25 883 it is known that, in particular under the conditions of a severely restricted kidney function and therefore necessary kidney replacement therapy in the form of haemodialysis or peritoneal dialysis, the cellular thiol-disulphide metabolism is badly disturbed. This disturbance results inter alia in an extensive loss of normal cell functions, that of the capacity of peritoneal macrophages for phagocytosis or the capacity of lymphocytes to be activated.

DE 101 25 832 describes studies within the framework of diabetes mellitus, in which a displacement of the redox state on account of reduced glutathione as well as an absolute reduction in the total pool of glutathione was able to be proved. This defect can be removed by a combination of α-lipoic acid and prothiols.

Proceeding from the above, it was the object of the present invention to make available a medicine with which the functionality of the alveolar macrophages can be restored in chronically obstructive lung diseases and the connected defect in the homeostasis of the cytokine production can be eliminated.

This object is accomplished by the utilisation having the features of claim 1. The additional dependent claims disclose advantageous developments.

According to the invention, the use of at least one effector of glutathione metabolism together with α-lipoic acid, its salts and/or its pro-drugs for the simultaneous, separate or timed cytoprotective treatment of chronically obstructive lung diseases is taught.

It was able to be demonstrated that, due to the application of the inventive combination of α-lipoic acid and the effectors of glutathione metabolism, there was a normalisation of the primarily reduced thiol status of alveolar macrophages. Not only did the thiol-stabilising effect of the combination regularly exceed that of solely using α-lipoic acid or the respective effectors, but superadditive effects could also be demonstrated.

The restoration of the thiol status included here both intracellular thiols and membrane-bound SH groups and thus is an expression of a complex biological regulation. This phenomenon is based on the fact that the effectors of glutathione metabolism on the one hand eliminate intermediately produced free radicals and on the other hand increase the availability of reducing equivalents for the conversion of the α-lipoic acid from disulphide form to reduced form and thus improve the synthesis-inducing effect of the α-lipoic acid on the thiol-disulphide status. The restoration of the thiol status of the immune cells was accompanied by a normalisation of the phagocytosis activity as an expression of a regulation of central functional parameters.

The combinations of α-lipoic acid with an effector of glutathione metabolism, used according to the invention, can be administered in the conventional pharmacological forms or as an instillate both prophylactically and therapeutically. The effective dose has to be determined according to the individual case and preferably lies in the range between 30 and 1800 mg/d and by particular preference between 200 and 600 mg/d α-lipoic acid.

Preferably ambroxol of the general formula I 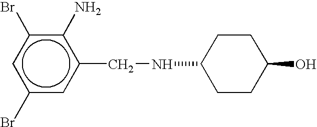
its salts and/or its pro-drugs is/are used as the effector of glutathione metabolism. The dose of ambroxol, its salts and/or its pro-drugs for administration to a human patient is preferably in the range between 7.5 and 90 mg/d and by particular preference between 60 and 75 mg/d.

Ambroxol is used in various administration forms for lung and bronchial diseases as a mucolytic medicine. The effect of ambroxol as a mucolytic agent is based both on a stimulation of the suffactant production of the bronchial cells and, particularly, on the capability of eliminating free radicals. The anti-oxidative activity, based on this, of the substance was able to be proved mainly on pulmonary cells but also within the framework of inflammatory mechanisms.

In a further preferred variant, silibinin is used as the effector of glutathione metabolism. The dose of silibinin, its salts and/or its pro-drugs for administration to a human patient is preferably between 20 and 1600 mg/d and by particular preference between 300 and 800 mg/d.

Silibinin is a representative of naturally occurring polyhydroxyphenyl chromanone compounds which are known as flavolignans. Within the group of flavolignans, silymarin is described as the extract of certain fruits. Silymarin is in turn a complex of the flavenoids silibinin, silichristin, silidianin and bitter constituents. As described in DE 35 37 656, silibinin including the positional isomers can be isolated from this complex.

The described medicine can be administered by inhalation, orally or parenterally. The medicine can be in the form of an aerosol, for example as a dust or mist aerosol, a spray or inhalation aerosol, a solution, granules, a powder, an emulsion, a tablet and/or a film tablet.

The medicine can preferably contain further additives selected from the group of aqueous solvents, stabilisers, suspending, dispersing and wetting agents.

The effector of glutathione metabolism and the α-lipoic acid can be presented both in a single formulation and in separate formulations.

The utilisation according to the invention will now be explained in greater detail with the aid of the following examples.

### EXAMPLE 1

Influence of the combination of α-lipoic acid with ambroxol on the cellular thiol status of alveolar macrophages

The established normal alveolar macrophage cell line CRL 21-92 (NR8383 [AgC11x3A; NR8383.1]) was used. The cells were taken up in special cell culture media and incubated in a gassing incubator at 37° C., a relative air humidity of 98% and a relative air-CO₂ content of 5%. In order to check the influence of the combinations used according to the invention on the thiol status of thiol-deficient alveolar macrophages, these were artificially thiol-depleted. This came about by cultivation in thiol-deficient media (TDM) according to tested methods [Free Radic Biol Med 2000; 29:1160-1165]. Comparative cultures using complete media (RPMT 1640) were used for defining the best possible normal value under culture conditions.

The intercellular thiol content at the individual cell level was determined using 5-chloromethylfluorescein diacetate (CMFDA) in flow cytofluorimetry [Cytometry 1994; 15:349-358, Cytometry 1997; 29:76-82].

CMFDA which is primarily non-fluorogenic is here passively absorbed by the cell. Via the chloromethyl residue there is binding to cytoplasmatic thiol groups. After the acetate residues have been split off by unspecific cellular esterases, this complex which is now cell membrane-impermeable becomes fluorogenic at an excitation wavelength of λₑₓ=490 nm with an emission wavelength of λₑₘ=520 nm. The mean fluorescence intensity of the sample (10,000 cells) is directly proportional to the concentration of the intracellular thiol groups.

The expression of membrane-bound thiol groups was also determined by means of flow cytofluorimetry. Chloromethyl tetramethyl rhodamine (CMTMR) was used as the thiol conjugate under conditions of a blocked membrane potential and an inhibited diffusion capacity of the cells, [Exp Hematol 1997; 25(7): 601-607]. The fluorescence intensity of the bound fluorochrome molecules on the cell membrane is here again proportional to the quantity of thiol groups on the cell surface. Immortalised alveolar macrophages were artificially thiol-depleted in the above-described test assembly. The influence of the substances used according to the invention was checked over a period of 96 hours by measuring the intracellular thiol content and the membrane expression of thiols.

It was demonstrated that, starting after 24 hours, the combination of α-lipoic acid and ambroxol in a wide concentration range from 100 nM to 10 μM initiated a complete restoration of the thiol status of the alveolar macrophages. As shown in Table 1, significant increases in the thiol content were demonstrated over the entire test period for these ambroxol doses. The effect of the α-lipoic acid alone was regularly significantly exceeded.

Table 1 shows the influence of α-lipoic acid in combination with ambroxol on the intracellular thiol concentration of an alveolar macrophage cell line [*: p<0.05, ANOVA, n=12].

TABLE 1

| Thiols, intracellular | 24 h | 48 h | 72 h | 96 h |
|---|---|---|---|---|
| (CMFDA)[%] | MW ± SD | MW ± SD | MW ± SD | MW ± SD |
| KO, normal | 100.0 ± 38.0 | 100.0 ± 67.9 | 100.0 ± 24.2 | 100.0 ± 5.4 |
| RPMI1640 | | | | |
| KO, thiol-deficient | 65.0 ± 6.0 | 35.7 ± 13.3 | 59.6 ± 16.8 | 73.3 ± 12.4 |
| TDM | | | | |
| αLA[10.0 μg/ml] | 70.2 ± 8.4 | 58.9 ± 10.4 | 78.1 ± 30.0 | 72.5 ± 19.1 |
| TDM | | | | |
| αLA[10.0 μg/ml] | 93.6 ± 17.8* | 96.9 ± 63.6* | 117.8 ± 12.5* | 102.7 ± 27.7* |
| Ambroxol [0.1 μM] | | | | |
| TDM | | | | |
| αLA[10.0 μg/ml] | 91.7 ± 8.6* | 77.5 ± 32.0* | 101.5 ± 26.8* | 101.1 ± 8.3* |
| Ambroxol [1.0 μM] | | | | |
| TDM | | | | |
| αLA[10.0 μg/ml] | 80.6 ± 9.7* | 71.8 ± 32.0* | 115.6 ± 25.8* | 101.0 ± 7.1* |
| Ambroxol [10.0 μM] | | | | |
| TDM | | | | |
| αLA[10.0 μg/ml] | 70.3 ± 6.7* | 74.6 ± 30.1* | 101.4 ± 23.1* | 62.4 ± 15.9* |
| Ambroxol [100.0 μM] | | | | |
| TDM | | | | |
| αLA[10.0 μg/ml] | 10.5 ± 7.3 | 40.7 ± 24.5 | 22.5 ± 5.2 | 20.5 ± 21.5 |
| Ambroxol [1000.0 μM] | | | | |
| TDM | | | | |

Table 2 shows the influence of α-lipoic acid in combination with ambroxol on the membrane thiol concentration of an alveolar macrophage cell line [*: p<0.05, ANOVA, n=12]. Table 2 is based on the parallel induction of membrane thiols.

TABLE 2

| Thiols, in membrane | 24 h | 48 h | 72 h | 96 h |
|---|---|---|---|---|
| (CMTMR)[%] | MW ± SD | MW ± SD | MW ± SD | MW ± SD |
| KO, normal | 100.0 ± 52.6 | 100.0 ± 7.9 | 100.0 ± 44.9 | 100.0 ± 44.2 |
| RPMI1640 | | | | |
| KO, thiol-deficient | 75.9 ± 38.2 | 86.1 ± 15.0 | 68.2 ± 3.3 | 82.7 ± 44.1 |
| TDM | | | | |
| αLA[10.0 μg/ml] TDM | 91.1 ± 66.3 | 165.5 ± 36.3 | 82.3 ± 42.1 | 118.9 ± 76.6 |
| αLA[10.0 μg/ml] | 90.7 ± 55.7 | 176.3 ± 6.1* | 82.5 ± 31.6 | 122.6 ± 76.8* |
| Ambroxol [0.1 μM] | | | | |
| TDM | | | | |
| αLA[10.0 μg/ml] | 96.2 ± 55.4 | 161.1 ± 25.3* | 82.2 ± 23.9* | 144.9 ± 110.4* |
| Ambroxol [1.0 μM] | | | | |
| TDM | | | | |
| αLA[10.0 μg/ml] | 97.3 ± 51.7 | 165.8 ± 37.2* | 87.2 ± 31.0* | 125.8 ± 73.4* |
| Ambroxol [10.0 μM] | | | | |
| TDM | | | | |
| αLA[10.0 μg/ml] | 106.6 ± 61.2* | 170.3 ± 36.8* | 93.5 ± 28.9* | 117.0 ± 51.0* |
| Ambroxol [100.0 μM] | | | | |
| TDM | | | | |
| αLA[10.0 μg/ml] | 110.7 ± 25.6 | 159.9 ± 18.2* | 95.1 ± 27.5 | 125.0 ± 15.7* |
| Ambroxol [1000.0 μM] | | | | |
| TDM | | | | |

### EXAMPLE 2

Influence of α-lipoic acid in combination with silibinin on the cellular thiol status of alveolar macrophages

Immortalised alveolar macrophages were artificially thiol-depleted in the test assembly described in Example 1. The influence of the substances used according to the invention was checked over a period of 96 hours by measuring the intracellular thiol content and the membrane expression of thiols.

It was demonstrated that, starting after 24 hours, the combination of α-lipoic acid and silibinin in a narrow concentration range around 70 μg/ml initiated a complete restoration of the thiol status of the alveolar macrophages. As shown in Table 3, significant increases in the thiol content were demonstrated over the entire test period for this silibinin dose. The effect of the α-lipoic acid alone was regularly significantly exceeded. Smaller additive concentrations of silibinin also induced a thiol regeneration after treatment of a longer duration.

The influence of membrane thiols was, as recorded in Table 4, demonstrable in the above-mentioned concentration-time regimes. In contrast to intracellular induction, surface thiols were already modulated after 24 hours in the presence of smaller concentrations of silibinin.

Table 3 shows the influence of α-lipoic acid in combination with silibinin on the intracellular thiol concentration of an alveolar macrophage cell line [*: p<0.05, ANOVA, n=12].

TABLE 3

| Thiols, intracellular | 24 h | 48 h | 72 h | 96 h |
|---|---|---|---|---|
| (CMFDA)[%] | MW ± SD | MW ± SD | MW ± SD | MW ± SD |
| KO, normal | 100.0 ± 29.0 | 100.0 ± 29.3 | 100.0 ± 52.4 | 100.0 ± 14.2 |
| RPMI1640 | | | | |
| KO, thiol-deficient | 77.4 ± 26.6 | 86.8 ± 4.0 | 88.8 ± 50.0 | 82.1 ± 13.7 |
| TDM | | | | |
| αLA[10.0 μg/ml] | 69.2 ± 28.2 | 91.1 ± 16.6 | 88.6 ± 26.2 | 114.8 ± 33.2 |
| TDM | | | | |
| αLA[10.0 μg/ml] | 77.5 ± 26.8* | 91.5 ± 17.3 | 92.9 ± 45.0 | 122.6 ± 33.1* |
| Silibinin [0.07 μg/ml] | | | | |
| TDM | | | | |
| αLA[10.0 μg/ml] | 75.1 ± 27.4 | 93.1 ± 24.7 | 90.0 ± 37.8 | 108.2 ± 22.9* |
| Silibinin [0.7 μg/ml] | | | | |
| TDM | | | | |
| αLA[10.0 μg/ml] | 73.3 ± 2.3 | 90.4 ± 29.0 | 85.9 ± 35.7 | 124.2 ± 40.1* |
| Silibinin [7.0 μg/ml] | | | | |
| TDM | | | | |
| αLA[10.0 μg/ml] | 161.7 ± 76.7* | 173.4 ± 85.2* | 126.6 ± 29.5* | 143.3 ± 51.9* |
| Silibinin [70.0 μg/ml] | | | | |
| TDM | | | | |
| αLA[10.0 μg/ml] | 40.9 ± 17.4 | 18.7 ± 10.5 | 17.1 ± 11.6 | 22.5 ± 7.5 |
| Silibinin [700.0 μg/ml] | | | | |
| TDM | | | | |

Table 4 shows the influence of α-lipoic acid in combination with silibinin on the membrane thiol concentration of an alveolar macrophage cell line [*: p<0.05, ANOVA, n=12].

TABLE 4

| Thiols, in membrane | 24 h | 48 h | 72 h | 96 h |
|---|---|---|---|---|
| (CMFDA)[%] | MW ± SD | MW ± SD | MW ± SD | MW ± SD |
| KO, normal | 100.0 ± 6.8 | 100.0 ± 8.2 | 100.0 ± 34.5 | 100.0 ± 47.0 |
| RPMI1640 | | | | |
| KO, thiol-deficient | 82.5 ± 3.6 | 88.9 ± 7.3 | 87.5 ± 35.7 | 100.2 ± 54.5 |
| TDM | | | | |
| αLA[10.0 μg/ml] | 108.8 ± 38.2 | 126.2 ± 68.6 | 90.7 ± 28.1 | 107.3 ± 57.4 |
| TDM | | | | |
| αLA[10.0 μg/ml] | 103.4 ± 54.6 | 126.7 ± 56.8 | 106.3 ± 39.5 | 109.9 ± 60.9 |
| Silibinin [0.07 μg/ml] | | | | |
| TDM | | | | |
| αLA[10.0 μg/ml] | 124.3 ± 21.7* | 133.8 ± 54.2* | 91.8 ± 36.6 | 110.9 ± 45.9 |
| Silibinin [0.7 μg/ml] | | | | |
| TDM | | | | |
| αLA[10.0 μg/ml] | 109.4 ± 32.9* | 175.2 ± 65.8* | 135.7 ± 21.0* | 111.2 ± 30.4 |
| Silibinin [7.0 μg/ml] | | | | |
| TDM | | | | |
| αLA[10.0 μg/ml] | 150.0 ± 24.1* | 138.7 ± 62.4* | 102.0 ± 45.0* | 110.5 ± 44.2 |
| Silibinin [70.0 μg/ml] | | | | |
| TDM | | | | |
| αLA[10.0 μg/ml] | 68.3 ± 4.0 | 103.1 ± 26.1 | 91.1 ± 32.9 | 122.1 ± 39.2 |
| Silibinin [700.0 μg/ml] | | | | |
| TDM | | | | |

### EXAMPLE 3

Influence of the combination of α-lipoic acid in combination with ambroxol on the cellular thiol status of primary alveolar macrophages of COPD patients

Alveolar macrophages were isolated from the bronchoalveolar lavage fluid of COPD patients, taken up in cell culture medium and incubated in a gassing incubator at 37° C., a relative air humidity of 98% and a relative air-CO₂ content of 7.5%. In order to check the influence of the combinations used according to the invention on the thiol status of the peritoneal macrophages, in each case one fraction was treated with α-lipoic acid, the effector of glutathione metabolism ambroxol or with the combination of α-lipoic acid/ambroxol, whilst another fraction was used in each case as an untreated control. Untreated alveolar macrophages from healthy, non-COPD patients served as the normal comparison.

The cellular thiol status was determined by means of the measuring method described under 1. The effect of the combination of α-lipoic acid and ambroxol in time kinetics over 96 hours in relation to healthy cells is shown in Table 5.

With the addition of the monosubstances, only a marginal rise in cellular thiol expression was observed using ambroxol, whilst α-lipoic acid showed no effect. In contrast, with the combination of α-lipoic acid and ambroxol, a clear rise in cellular thiol expression could be demonstrated, starting after 24 hours, which reached a superadditive and significant maximum over the entire test period in the presence of 10 μM ambroxol.

Table 5 shows the influence of α-lipoic acid in combination with ambroxol on the cellular thiol concentration of primary alveolar macrophages of COPD patients [*: p<0.05, ANOVA, n=8].

TABLE 5

| Thiols, intracellular | 24 h | 48 h | 72 h | 96 h |
|---|---|---|---|---|
| (CMFDA)[%] | MW ± SD | MW ± SD | MW ± SD | MW ± SD |
| Control, normal | 100.0 ± 19.7 | 100.0 ± 13.1 | 100.0 ± 31.8 | 100.0 ± 23.2 |
| RPMI1640 | | | | |
| Control, COPD. | 61.6 ± 13.9 | 58.6 ± 13.7 | 46.1 ± 18.8 | 51.9 ± 14.3 |
| RPMI1640 | | | | |
| αLA[10.0 μg/ml] | 60.3 ± 21.0 | 58.0 ± 17.0 | 46.2 ± 19.8 | 62.4 ± 6.3 |
| RPMI1640 | | | | |
| Ambroxol [10.0 μ/ml] | 67.4 ± 16.4 | 61.8 ± 11.5 | 61.6 ± 20.8 | 67.1 ± 8.4 |
| RPMI1640 | | | | |
| αLA[10.0 μg/ml] | 81.1 ± 15.7* | 81.9 ± 10.1* | 83.9 ± 18.2* | 81.2 ± 8.8* |
| Ambroxol [1.0 μM] | | | | |
| RPMI1640 | | | | |
| αLA[10.0 μg/ml] | 101.2 ± 17.7* | 109.5 ± 13.4* | 113.9 ± 25.6* | 107.8 ± 26.8* |
| Ambroxol [10.0 μM] | | | | |
| RPMI1640 | | | | |
| αLA[10.0 μg/ml] | 84.7 ± 25.5 | 79.6 ± 13.4 | 70.0 ± 31.8* | 76.6 ± 9.7 |
| Ambroxol [100.0 μM] | | | | |
| RPMI1640 | | | | |
| αLA[10.0 μg/ml] | 31.5 ± 12.1 | 53.8 ± 22.8 | 34.8 ± 2.1 | 36.1 ± 4.2 |
| Ambroxol [1000.0 μM] | | | | |
| RPMI1640 | | | | |

### EXAMPLE 4

Influence of the combination of α-lipoic acid in combination with silibinin on the cellular thiol status of primary alveolar macrophages of COPD patients

In a test assembly identical to that of Example 3, alveolar macrophages from COPD patients were in each case treated with α-lipoic acid, the effector silibinin or with the combination of α-lipoic acid/silibinin, whilst another fraction was again used in each case as an untreated control. Untreated alveolar macrophages from healthy, non-COPD patients served here, too, as the normal comparison.

The cellular thiol status was determined by means of the measuring method described under 1. The effect of the combination of α-lipoic acid and silibinin in time kinetics over 96 hours in relation to healthy cells is shown in Table 6.

With the addition of the monosubstances α-lipoic acid or silibinin, no modulation of the cellular thiol expression was to be observed. In contrast, with the combination of α-lipoic acid and silibinin, a clear rise in cellular thiol expression could be demonstrated, starting after 24 hours, which reached a superadditive and significant maximum over the entire test period in the presence of 70 μg/ml silibinin.

Table 6 shows the influence of α-lipoic acid in combination with silibinin on the cellular thiol concentration of primary alveolar macrophages of COPD patients [*: p<0.05, ANOVA, n=8].

TABLE 6

| Thiols, intracellular | 24 h | 48 h | 72 h | 96 h |
|---|---|---|---|---|
| (CMFDA) [%] | MW ± SD | MW ± SD | MW ± SD | MW ± SD |
| Control, normal | 100.0 ± 19.7 | 100.0 ± 8.2 | 100.0 ± 31.8 | 100.0 ± 23.2 |
| RPMI1640 | | | | |
| Control, COPD, | 61.6 ± 13.9 | 58.6 ± 7.3 | 46.1 ± 18.8 | 51.9 ± 14.3 |
| RPMI1640 | | | | |
| αLA [10.0 μg/ml] | 60.3 ± 21.0 | 58.0 ± 68.6 | 46.2 ± 19.8 | 62.4 ± 6.3 |
| RPMI1640 | | | | |
| Silibinin [70 μg/ml] | 56.1 ± 12.4 | 59.0 ± 56.8 | 44.7 ± 14.0 | 49.4 ± 14.5 |
| RPMI1640 | | | | |
| αLA[10.0 μg/ml] | 64.1 ± 10.2* | 64.7 ± 54.2* | 49.9 ± 11.8 | 62.6 ± 8.0 |
| Silibinin [0.7 μg/ml] | | | | |
| RPMI1640 | | | | |
| αLA[10.0 μg/ml] | 84.5 ± 14.1* | 76.0 ± 65.8* | 78.6 ± 14.9* | 81.8 ± 17.8 |
| Silibinin [7.0 μg/ml] | | | | |
| RPMI1640 | | | | |
| αLA[10.0 μg/ml] | 102.5 ± 22.6* | 103.3 ± 62.4* | 100.0 ± 27.1* | 92.7 ± 20.1* |
| Silibinin [70.0 μg/ml] | | | | |
| RPMI1640 | | | | |
| αLA[10.0 μg/ml] | 59.4 ± 11.7 | 36.2 ± 26.1 | 38.8 ± 10.3 | 35.7 ± 2.7 |
| Silibinin [700.0 μg/ml] | | | | |
| RPMI1640 | | | | |

### EXAMPLE 5

Influence on the capacity of alveolar macrophages for phagocytosis

In order to make possible a characterisation of the alveolar macrophages in respect of their original functions, the capacity for phagocytosis was selected as the measured variable.

In a similar procedure to the one described in Example 3, alveolar macrophages were isolated and cultivated ex vivo. The phagocytosis efficiency was determined by a cytofluorimetric test at the single-cell level. The macrophages were co-cultivated with opsonized and fluorochrome-marked bacteria. The amount of bacteria absorbed in a defined period of time was assessed quantitatively via the fluorescence intensity in the macrophages and counted as the measurement for their capacity for phagocytosis. The influence of the combinations used according to the invention on the capacity of the peritoneal macrophages for phagocytosis after a treatment period of up to 96 hours is shown in Table 7.

After incubation with α-lipoic acid, ambroxol or silibinin, the phagocytosis rate had not altered by comparison with the untreated control. On the other hand, using the combination of α-lipoic acid and ambroxol, a significant increase in the phagocytosis rate could be achieved which corresponded after 72 hours to the values of the healthy control group.

The course of the induction of phagocytosis with the combination of α-lipoic acid and silibinin in a concentration of 70 μg/ml was similar. Here, too, a significant improvement in the capacity for phagocytosis was demonstrated, parallel to a restoration of the thiol status.

Table 7 shows the influence of α-lipoic acid in combination with ambroxol or silibinin on the phagocytosis rate of primary alveolar macrophages of COPD patients [*: p<0.05, ANOVA, n=6].

TABLE 7

| Phagoburst | 24 h | 48 h | 72 h | 96 h |
|---|---|---|---|---|
| (mfi) [%] | MW ± SD | MW ± SD | MW ± SD | MW ± SD |
| Control, normal | 100.0 ± 16.8 | 100.0 ± 22.2 | 100.0 ± 14.6 | 100.0 ± 13.7 |
| RPMI1640 | | | | |
| Control, COPD, | 56.9 ± 15.5 | 55.4 ± 14.1 | 57.0 ± 9.7 | 67.7 ± 13.7 |
| RPMI1640 | | | | |
| αLA [10.0 μg/ml] | 59.0 ± 11.7 | 60.2 ± 12.4 | 50.7 ± 9.0 | 64.9 ± 14.3 |
| RPMI1640 | | | | |
| Ambroxol [10 μM] | 53.3 ± 10.9 | 59.2 ± 12.4 | 55.2 ± 19.2 | 61.9 ± 22.1 |
| RPMI1640 | | | | |
| Silibinin [70.0 μg/ml] | 55.1 ± 12.2* | 57.6 ± 8.5 | 54.2 ± 13.8 | 59.9 ± 21.2 |
| RPMI1640 | | | | |
| αLA [10.0 μg/ml] | 81.5 ± 19.0* | 93.2 ± 18.4* | 116.4 ± 17.6* | 102.8 ± 4.8* |
| Ambroxol [10 μM] | | | | |
| RPMI1640 | | | | |
| αLA [10.0 μg/ml] | 75.5 ± 17.3* | 86.5 ± 16.7* | 98.7 ± 22.6* | 92.5 ± 9.1* |
| Silibinin [70.0 μg/ml] | | | | |
| RPMI1640 | | | | |

Overall, these tests make it clear that the application of the combination of α-lipoic acid and the effectors of glutathione metabolism, ambroxol or silibinin, stabilises a primarily massively damaged thiol status in thiol-deficient alveolar macrophages both after artificial thiol deficiency and in COPD patients. This normalisation leads furthermore to a restoration of central cellular functions, such as phagocytosis activity, which is not to be recorded without such treatment.

1. Use of at least one effector of glutathione metabolism together with α-lipoic acid, its salts and/or its pro-drugs for the simultaneous, separate or timed cytoprotective treatment of chronically obstructive lung diseases.

2. Use according to claim 1, characterised in that the dose of the α-lipoic acid, its salts and/or its pro-drugs for administration to a human patient is between 30 and 1800 mg/d, preferably between 200 and 600 mg/d.

3. Use according to at least one of the preceding claims,
characterised in that ambroxol of the general formula I, 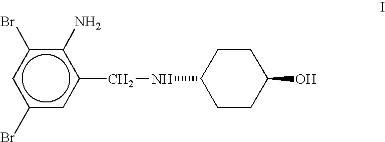
its salts and/or its pro-drugs is/are used as the effector.

4. Use according to claim 3,
characterised in that the dose of ambroxol, its salts and/or its pro-drugs for administration to a human patient is between 7.5 and 90 mg/d, preferably between 60 and 75 mg/d.

5. Use according to at least one of the preceding claims, characterised in that silibinin, its salts and/or its pro-drugs is/are used as the effector.

6. Use according to claim 5,
characterised in that the dose of silibinin, its salts/or its pro-drugs for administration to a human patient is between 20 and 1600 mg/d, preferably between 300 and 800 mg/d.

7. Use according to at least one of the preceding claims,
characterised in that the medicine can be administered by inhalation, orally or parenterally.

8. Use according to at least one of the preceding claims,
characterised in that the medicine contains further additives selected from the group of aqueous solvents, stabilisers, suspending, dispersing and wetting agents.

9. Use according to at least one of the preceding claims,
characterised in that the medicine is presented in the form of an aerosol, a solution, granules, a powder, an emulsion, a tablet and/or a film tablet.

10. Use according to at least one of the preceding claims,
characterised in that the effector of glutathione metabolism and the α-lipoic acid, its salts and/or its pro-drugs are presented in a single formulation.

11. Use according to at least one of the preceding claims,
characterised in that the effector of glutathione metabolism and the α-lipoic acid, its salts and/or its pro-drugs are presented in separate formulations.
